(19) United States
(12) Patent Application Publication
(10) Pub. No.: US 2022/0199229 A1
(43) Pub. Date: Jun. 23, 2022

(54) METHOD AND SYSTEM FOR ENHANCING MEDICAL ULTRASOUND IMAGING DEVICES WITH COMPUTER VISION, COMPUTER AIDED DIAGNOSTICS, REPORT GENERATION AND NETWORK COMMUNICATION IN REAL-TIME AND NEAR REAL-TIME

(71) Applicant: Wavebase Inc., Waterloo (CA)

(72) Inventors: Peter Nikolai Holmes, Kitchener (CA); Jason Lars Deglint, Waterloo (CA); James Andrew Stone, Victoria (CA); Ahmed Gawish, Waterloo (CA)

(21) Appl. No.: 17/541,233

(22) Filed: Dec. 2, 2021

Related U.S. Application Data
(60) Provisional application No. 63/120,830, filed on Dec. 3, 2020.

(51) Int. Cl. G16H 30/20; G16H 40/63; G16H 50/20; G16H 10/60; G06F 3/0488

(52) U.S. Cl. CPC G16H 30/20; G16H 40/63; G06F 3/0488; G16H 10/60; G16H 50/20

(57) ABSTRACT

This method and system supplements medical ultrasound imaging devices with a user installable touchscreen monitor and processor that delivers additional functionality to the device. This addition allows for multiple machine learning models working in sequence to provide real-time and near real-time new information to the user. This new information is being produced by multiple machine learning models that are installed on the device and selected by the user using the touch screen to identify features, perform computer aided diagnostics, automatic image calibration, automatically highlight regions of interest, feature highlighting, automatic annotation of images and report generation. The data gathered from the image processing and user inputs are then converted to an industry standard data format. This new information is combined into a report for user review and transferred to the hospital picture archive and communication system, the electronic health record and the hospital billing system.

Claims Benefit of Provisional Patent Application No. 63/120,830 Filed 3 Dec. 2020 with the same title as above.

### TECHNICAL FIELD

The present subject matter relates generally to medical imaging technologies and particularly to a system for enhancing accuracy in capturing and reporting of ultrasound studies using in real-time and near real-time.

### BACKGROUND

Over the past few decades, we have witnessed a dramatic rise in life expectancy owing to significant advances in medical science and technology, medicine as well as increased availability of medical tests and accurate diagnosis of diseases. With such improvisation, research into diagnosis technologies has gained prominence over the recent years.

Medical imaging is an important diagnosis technology that supports the entire clinical imaging workflow from diagnosis, patient stratification, therapy planning, intervention, and follow-up. Medical image identification and classification refers to the detection of boundaries of structures, such as organs, vessels, different types of tissue, pathologies, medical devices, etc., in medical images of a patient. Examples of various medical imaging technologies include computed tomography (CT), magnetic resonance imaging (MM), ultrasound, x-ray, DynaCT, positron emission tomography (PET), laparoscopic/endoscopic imaging, etc. among others. Among these ultrasound devices have recently gained a lot of exposure due to its safety, minimal side effects, and utility for real-time bedside diagnosis in the emergency room and Intensive Care Unit.

Conventionally, ultrasound systems are large, complex, and expensive systems that are typically used in large medical facilities (such as a hospital) and are operated by medical professionals that are experienced with these systems, such as ultrasound technicians. Ultrasound technicians typically undergo years of hands-on training to learn how to properly use the ultrasound imaging system. For example, an ultrasound technician may learn how to appropriately position an ultrasound device on a subject to capture an ultrasound image in various anatomical views (Image acquisition). Further, an ultrasound technician may learn how to read captured ultrasound images to infer medical information about the patient and/or those images may be sent to a radiologist for later review (Image Interpretation).

Recently, such medical imaging technologies have become smaller and more portable which has led to a rise in point of care imaging which allows for faster clinical decisions without waiting for patient transfer to a radiology lab or imaging clinic. This reduction in cost and size allows many more healthcare workers to use this technology. In order to assist these new users of medical imaging devices, computer vision applications have been developed to aid with image acquisition guidance and computer aided diagnostics. While such tools are helpful, due to the lack of expertise of the users who are not trained in imaging technologies, accuracy in diagnosis/analysis of such has been a common problem.

Therefore, an improved automatic/guided acquisition using such medical imaging technologies have now become a prerequisite for many medical image analysis tasks, such as correct probing, disease diagnosis, and classification of the images. Further, diagnosis and/or classification without a physical presence of an expert, for such on-the go captured medical images needs to be improved too

Accordingly, it is desirable to provide a system that could accurately and efficiently work with a variety of ultrasound devices to enable a correct image acquisition and also provide a timely diagnosis of diseases using such medical images, to determine a patient's health status in a real-time and also be able to provide alerts and/or generate a computer aided diagnostic report.

The status quo for computer aided diagnostics involves sending those images to a third-party server either on premise or in the cloud for computer evaluation and subsequent human review at a later time. However, this process can take hours or even days for the doctor or radiologist to review the images. In the emergency room and intensive care unit waiting hours for a diagnosis can be life threatening.

Some newer ultrasound devices have some built in computer vision and computer aided design features. However, due to ultrasound devices being upgraded typically on a 7-10 year cycle an approach is necessary to speed up development and deployment of real time AI for ultrasound that does not require replacement of the ultrasound machine so that these new AI methods can be more effectively researched and tested without expensive hardware upgrades.

### SUMMARY

In one aspect of the present disclosure, a system for enhancing accuracy in acquisition and reporting using one or more ultrasound devices, in a real-time is disclosed. The system comprises of one or more ultrasound devices, each configured to be used for capture one or more images and/or video stream, of a target area. The system further includes a controller adapted to be connected to each of the ultrasound devices using one or more connection medium. The controller is generally a computing device having a first communication interface adapted to receive one or more ultrasound images from one or more ultrasound devices, and one or more medical information related to the patient from one or more sources. The controller further includes a first processor and a first memory configured to execute one or more first programming instructions embodied thereon. The first memory includes one or more pixel-tables adapted to store pixel entries for one or more predetermined features of the received images, each corresponding to a specific scanning depth. The controller further includes a plurality of predetermined analysis algorithms adapted to be processed by the first processor, each adapted to process the image and/or frame[s] thereof to provide an analysis thereof. The controller furthermore includes a reporting unit provided in the form of a display unit adapted to display an output of the analysis using one or more predetermined algorithms. The system furthermore includes a back-end server having a second processor and a second memory configured to execute one or more second programming instructions embodied thereon. The back-end server includes a data receiving component adapted to receive one or more datasets pertaining to the ultrasound images and/or frames thereof, from a plurality of controllers. The back-end server further includes a machine learning based image processing module adapted to upgrade each of the predetermined analysis models of the controllers. Particularly, the image acquisition module is configured to process the second programming instructions embodied onto the second memory in accordance with the data sets received from the plurality of controllers so as to train the predetermined analysis algorithms. In operation, the controller receives one or more ultrasound images from one or more ultrasound devices. Thereafter, the controller is configured to select one of a predetermined analysis algorithm, in accordance with the medical information of the patient and process the received ultrasound images to determine if the acquisition using the devices is correct and/or performing the analysis that includes extraction of pixels from the images, calibration thereof, and processing the same thereafter.

Generally, the medical information includes one or more information related to the patient, selected from one or more of but not limited to medical prescriptions, diagnostic guidelines, and diagnostic history of the patient.

Potentially, the medical information is in the form of a scanned input/images.

Further potentially, the controller includes an optical character recognizing component adapted to enable an extraction of information from the scanned medical information.

Particularly, the reporting unit includes one or more display units selected from but not limited to an interactive touch display unit, a LED Monitor, CRT Monitor, and any other suitable display unit known in the art.

Generally, the controller includes one or more second connection interface(s) for connecting the controller with the back-end server.

Potentially, the first connection interface and/or the second connection interface is a wired communication interface selected from one or more of but not limited to USB, HDMI, CSI, LAN, and the like.

Alternatively, the first connection interface and/or the second connection interface is a wireless communication interface selected from one or more of but not limited to wi-fi, Bluetooth, hotspot, internet, intranet, WLAN, and the like.

Optionally, the system includes a clamping arm adapted to support one or more ultrasound devices onto the controller and/or vice versa.

In another aspect of the present invention, a method for enhancing accuracy in acquisition and reporting using one or more ultrasound devices, in a real-time is disclosed. The method includes receiving one or more ultrasound images, from one or more ultrasound devices, at the controller. The method further includes receiving one or more medical information related to the patient, at the controller. Thereafter, the method includes processing the received ultrasound images and/or frame[s] thereof, by first selecting at least one predetermined analysis algorithm in accordance to the medical information, and implementing programming instructions embodied thereon to determine any correction needed in probe direction/orientation so as to capture the image correctly followed by analyzing the images by extracting pixels from the images, followed by calibrating if required, and processing thereof. The method further comprising visualizing the assessment onto a report generation component so as to display orientation correction guidelines and/or diagnosis of the received images.

Optionally, the method further includes a step of preprocessing the image[s] to determine a plurality of image frame[s] and or masking the images if required.

Further optionally, the method includes a step of image standardization before the step of preprocessing the images.

Potentially, the analysis of images and/or image frame[s] includes processing the images to perform a classification and/or identification/computer aided diagnosis functions thereupon.

Particularly, the method further includes storing the received images and/or frames within a central data repository of the controller.

Further, the method includes storing a plurality of extracted pixel details within a pixel table configured within the memory of the controller.

Potentially, the method includes upgrading each of the predetermined analysis model in accordance with a machine learning based image processing module at the back-end server.

Numerous additional features, embodiments, and benefits of the methods and apparatus of the present invention are discussed below in the detailed description which follows.

### BRIEF DESCRIPTION OF DRAWINGS

The accompanying drawings illustrate various embodiments of systems, methods, and other aspects of the disclosure. Any person having ordinary skill in the art will appreciate that the illustrated element boundaries (e.g., boxes, groups of boxes, or other shapes) in the figures represent one example of the boundaries. It may be that in some examples, one element may be designed as multiple elements or that multiple elements may be designed as one element. In some examples, an element shown as an internal component of one element may be implemented as an external component in another, and vice versa. Furthermore, elements may not be drawn to scale.

FIG. 1 is a system block diagram of an ultrasound imaging management system according to the present invention.

FIG. 2A is an exemplary ultrasound imaging management system in accordance with the present invention.

FIG. 2B is another exemplary ultrasound imaging management system in accordance with the present invention.

FIG. 2C is another exemplary ultrasound imaging management system in accordance with the present invention.

FIG. 3 is an exemplary back-end server in accordance with the present invention.

FIG. 4A is an exemplary visualization in accordance with the present invention.

FIG. 4B is an exemplary visualization of a portable ultrasound device in accordance with the present invention.

FIG. 5A is a flow chart illustrating a method of ultrasound imaging and/or diagnosing in real time, according to the present invention.

FIG. 5B is an exemplary flowchart illustrating a method of ultrasound imaging and/or diagnosing in real time, according to the present invention.

FIG. 5C is an exemplary flowchart illustrating a method of ultrasound imaging and/or diagnosing in real time, according to the present invention

FIG. 6A is a flow chart illustrating an exemplary method of image standardization, according to the present invention.

FIG. 6B is another flowchart illustrating an exemplary method of image standardization, according to the present invention.

FIG. 7 illustrates an exemplary environmental embodiment of ultrasound imaging management system, according to the present invention.

Various embodiments will hereinafter be described in accordance with the appended drawings, which are provided to illustrate, and not to limit the scope in any manner, wherein like designations denote similar elements, and in which:

### DETAILED DESCRIPTION

The present subject matter is best understood with reference to the detailed figures and description set forth herein. Various embodiments are discussed below with reference to the figures. However, those skilled in the art will readily appreciate that the detailed descriptions given herein with respect to the figures are simply for explanatory purposes as the methods and systems may extend beyond the described embodiments. For example, the teachings presented, and the needs of a particular application may yield multiple alternate and suitable approaches to implement the functionality of any detail described herein. Therefore, any approach may extend beyond the particular implementation choices in the following embodiments described and shown.

The present application discloses an ultrasound imaging management system for correctly capturing images and/or video streams, each comprising one or more image frames, even by an inexperienced user and further for diagnosing the captured images for one or more disease, and/or providing a remedial treatment in accordance with the diagnosis, without needing the presence of a technical expert to analyze the images. The system further enables visualizing such images and/or diagnosis on a reporting unit, preferably in a real time. The system is further adapted to auto generate various alarms to a medical practitioner and/or to take informed decisions such that they can be taken up for correction. The system is generally provided in combination with a graphically visualized client application that could be accessed with a computer device, preferably in the form of a mobile application on an appropriate mobile device such as tablet, smartphone etc. However, in another embodiment, the system may be in form of a web-based automated service accessible on a generally known computing unit.

Particularly, the system of the present subject matter is adapted to accurately capture and assess the target area of a patient's body while considering all the possible diagnosis factors in combination with medical information of the patients, which may remotely be utilized for the purpose of diagnosing any underlying disease and/or problem at the target area of the patient. Additionally, the system of the current disclosure enables machine learning and artificial intelligence-based probe orientation guidance to enable an accurate capture of area of interest on the patient's body, feature identification within the ultrasound images using one or more predetermined analysis and/or classification algorithms. Moreover, the system of current disclosure allows its user to save the images for future analysis as well as annotating them to compare with historical analysis, situation and/or conditions. Additionally, the system of current disclosure works on a multiple mode, a user input-based mode and/or an automatic mode where no manual input is required from the user. It is to be understood that unless otherwise indicated, this invention need not be limited to applications for ultrasound devices. As one of ordinary skill in the art would appreciate, variations of the invention may be applied to other medical imaging technologies such as x-rays, c-t scans, MRI, and the like. Moreover, the invention may be used in any other field of daily life where image capture and analysis are required. Moreover, it should be understood that embodiments of the present invention may be applied in combination with various other management systems such as hospital management, patient management, facility management systems, access management systems, human resource management system, occupational management systems, clinical systems, and the like, for various other possible applications. It must also be noted that, as used in this specification and the appended claims, the singular forms “a,” “an” and “the” include plural referents unless the context clearly dictates otherwise. Thus, for example, the term “a data-set” is intended to mean a single dataset or a combination of datasets, “an algorithm” is intended to mean one or more algorithms for the same purpose or a combination of algorithms for performing different program executions.

References to “one embodiment,” “an embodiment,” “at least one embodiment,” “one example,” “an example,” “for example,” and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in an embodiment” does not necessarily refer to the same embodiment.

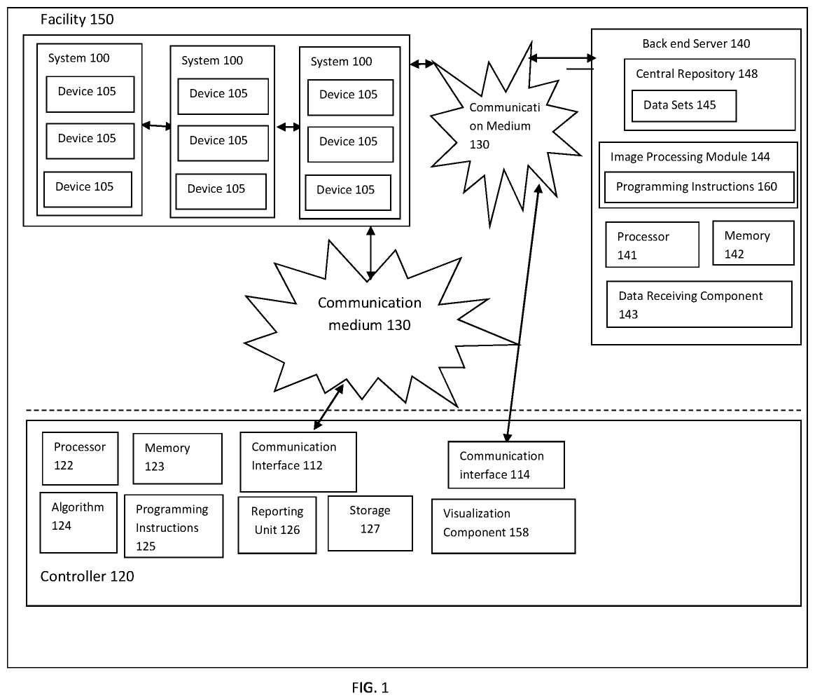

FIG. 1 is a system block diagram of an ultrasound imaging management system 100 according to the present invention adapted to capture and/or diagnosing a target area of a patient's body, within a facility 150. The system 100 includes one or more ultrasound devices 105, communicatively connected to a controller 120. It is to be contemplated for a person skilled in the art that a system environment can have any number of ultrasound devices 105 in accordance with the requirement within the facility 150 and may have multiple systems 100 connected to each other and to a backend server 140 through a communication medium 130.

The controller 120 includes a first processor 122, a first memory 123 and a plurality of predetermined analysis algorithms 124, each having one or more programming instructions 125 embodied thereon, adapted to be implemented by the first processor 122. The controller 120 further includes a reporting unit 126 provided in the form of a display unit, adapted to display an output of the analysis of images in accordance with one or more predetermined algorithms 124.

The controller 120 furthermore includes an inbuilt storage 127 to store the received images and/or processed analysis, which may also be pushed towards the backend server 140.

The controller 120 includes a first communication interface 112 for enabling a connection with one or more ultrasound devices 105 and a second communication interface 114 adapted to enable communication thereof with the back-end server 140 through the communication medium 130.

The first communication interface 112 is generally adapted to communicatively connect the ultrasound devices 105 to the controller 120. In a preferred embodiment, the first interface 112 is a low energy communication interface, generally in the form of Bluetooth, Infrared, and the like. In some embodiments, the first interface 112 is a high energy communication interface, generally in the form of a Wi-Fi interface, adapted to communicate with the ultrasound devices 105 through the communication medium 130, generally in the form a network selected from one or more of but not limited to a WAN, Internet, Intranet, other Cellular services (2G/4G or NB-IoT), and the like. However, in other embodiments, the communication interface 112 may be in the form of a wired interface such as USB, CSI, HDMI, LAN, and the like.

The second communication interface 114 is generally adapted to communicatively connect the controller 120 to the back-end server 140 through the communication medium 130. In a preferred embodiment, the second interface 114 is a high energy communication interface, generally in the form of a Wi-Fi interface, adapted to communicate with the back-end server 140 through the communication medium 130, generally in the form a network selected from one or more of but not limited to a WAN, Internet, Intranet, other Cellular services (2G/4G or NB-IoT), and the like. However, in other embodiments, the communication interface 114 may be in the form of a wired interface such as LAN, and the like.

The controller 120 furthermore includes one or more input units 117 selected from one or more of but not limited to one or more of a keyboard or keypad, a touchscreen or touch panel, a microphone, a mouse, a button, a remote control, a joystick, a telephone, or mobile device (e.g., a smartphone), a sensor, etc. Such input unit 117 are utilized to provide one or more input instructions from the users to the controller 120, particularly in embodiments, where the system 100 is operated under manual mode of operation. Such input units 117 is further used to receive the medical information related to the patient from the user and/or other sources

The back-end server 140 is generally a computing unit having a second processor 141, a second memory 142, one or more data-receiving component 143 adapted to receive datasets 145 from a plurality of controllers 120. The data-sets 145 pertains at least to a predetermined images and/or a medical information, received from one or more of plurality of controllers 120 and may also include other data-sets such as information from any out of the system sources such as industry compliant data like health level 7, patient's information from electronic health records, various picture archive and communication systems, and/or other ultrasound devices 105 directly, without connecting through any controller 120, health research institutes, expert opinions databases, and so on which may be helpful in improving the capture and/or diagnosis of the ultrasound images using one or more ultrasound devices 105.

The back-end server 140 further includes a central data repository 148 adapted to store the data sets 145 received at the data receiving component 143. In some embodiments, the central repository 148 is positioned within the back-end server 140 itself, as internal storage. In a preferred embodiment, the central repository 148 is remote to the back-end server 140 and works in a cloud-based environment. However, in other embodiments, the central repository 125 may be positioned in any possible configuration, as known in the art.

The back-end server 140 further includes a machine learning based image processing module 144 having a plurality of second programming instructions 160 embodied thereon to be implemented by the second processor 141. Particularly, the image processing module 144 is configured to process the received data sets 145 in accordance with one or more second programming instructions 160 so to determine a learning model that may compute and calculate deviation from expected values, based on the data-sets 145 processed in accordance to programming instructions 160 so as to calculate, identify, assess, rank, and determine a quantitative or qualitative value or level of diagnosis/decisions events based on known, anticipatory, historical, and/or premonitory data related to controller(s) 120 connected to the back-end server 140.

In an embodiment, the data repository 148 may include a decision-making database 165 comprising a plurality of decision data sets 167 such as for example, including but not limited to a plurality of probe angle control parameters and/or features dataset, diagnosis assessment reports comprising assessments of various images, target areas, decision dataset comprising suggested recommendations and/or remedial/treatment plan for overcoming said unwanted disease. The data sets 167 comprises a historical database from a plurality of different medical facilities, controllers, across various geographic and demographic regions, races, origins, socio-economic, biological considerations, and various other similar variations. In some embodiments, the remedial datasets may be collected from the external sources, such as health service providers and research institutes, and/or the management data accumulated by such health management institutes.

The data repository 148 including the decision database 165 and the plurality of datasets 145, 167 are constantly upgraded on the basis of one or more learning models selected from but not limited to Natural language processing (NLP), Deep Learning, Machine Learning, statistical learning model, and the like. Further, such imaging processing model 144 is further configured to upgrade each of the predetermined analysis algorithm 124 so as to improvise the image acquisition and/or diagnosis using the ultrasound devices 105, on the basis of deep learning developed by various datasets present within the data-repository 148.

In an embodiment of the present invention, the predetermined analysis algorithms 124, including the programming instructions 150 are based on a deep learning model wherein the model is particularly upgraded on the basis of datasets stored within the data repository 148, including received datasets 145, decision datasets 167 and the like.

Particularly, the deep learning model includes a number of pre-processing steps that are applied on the data stored in all the individual data sets 145, 167. The pre-processing steps may include cleansing the data to remove any inconsistencies and assigning weights to each of the parameters for the consideration of assessments. Particularly, a list of parameters/features may be determined at this step.

Further, the machine learning model and/or the deep learning model includes a learning engine adapted to run a selected model (e.g., deep learning model, Random Forest, multilinear regression, Multilayered, feed-forward neural networks, statistical model, or the like) on the data sets 145, 167, and partitions them into either a training dataset or a testing dataset. In a preferred embodiment, the partitioning may apply an 80/20 split between the training dataset and the testing dataset, respectively.

Thereafter, the learning engine operates to then run the selected model on the training dataset to obtain a resulting output from the model. For example, in a preferred embodiment, the selected model is the Multilayered, feed-forward neural networks, with a Tensor flow backend to build and train the neural networks.

The learning engine then selects and tunes other model arguments of the training dataset to establish an error percentage. Once the error percentage (i.e., accuracy) is established, the learning engine applies a ten-fold cross validation to establish model stability of the selected model. Further, the learning engine operates dynamically by dynamically selecting the model arguments for each run of the selected model.

Further, the learning engine operates a final model run on the testing dataset to confirm the accuracy and/or fit of the selected model are within client acceptable limits. When the accuracy and/or fit of the selected model is not within the client acceptable limits or when there are more models left for consideration, a next model may be selected to begin the testing process over again. When the accuracy and/or fit of the selected model is determined to be within the client acceptable limits or when there are no more models left for consideration, the selected model is established for use to predict probe angles/image acquisitions and/or analysis for the images received from one or more ultrasound devices 105.

In certain other embodiments, the second programming instructions 160 may be based on any predetermined medical image analysis models selected from a statistical models (e.g., linear regression, non-linear regression, Monte Carlo simulation), heuristic models (e.g., neural networks, fuzzy logic models, expert system models, state vector machine models useful in risk and safety prediction), and so on, that may be used to predict any problems within image capture/disease diagnosis/remedial plans, well in advance within the facility 150.

In some embodiments, the back-end server 140 further includes an informed decision module 149 adapted to utilize the programming instruction set 160 to generate a decision plan adapted to reduce the chances of faulty capture/wrong diagnosis/errors in analysis of the image received from the ultrasound devices 105, within the facility 150. Further, such an informed decision module 128 in accordance with informed decision datasets 167 is adapted to provide a plurality of recommendations and/or suggestions suitable for properly orienting various ultrasound devices 105/usage at a correct angle so as to avoid faulty capture of the images.

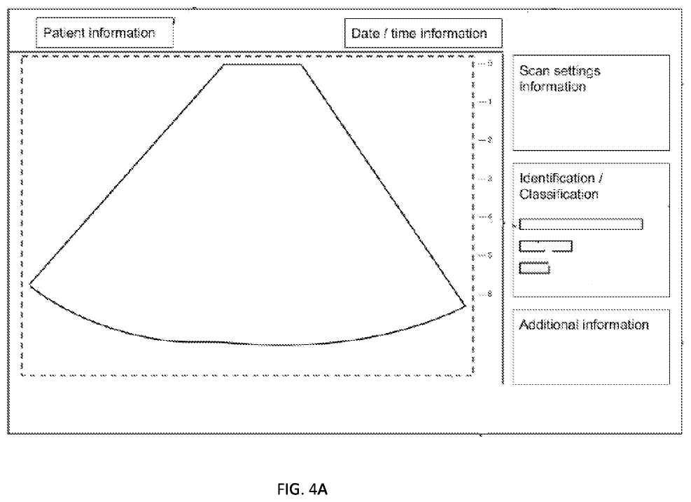

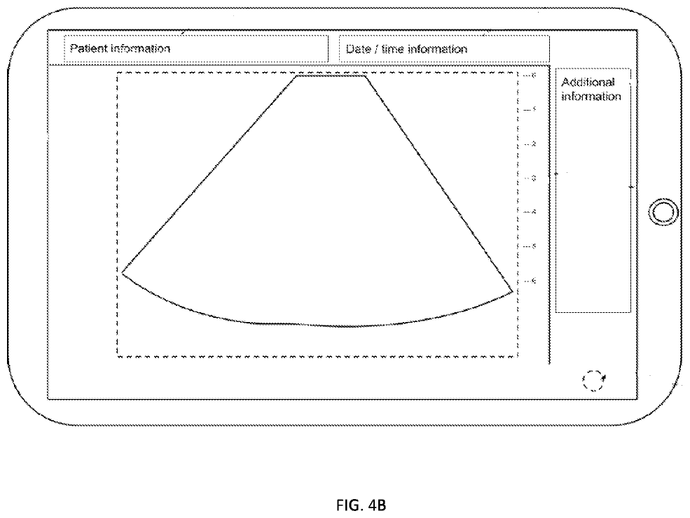

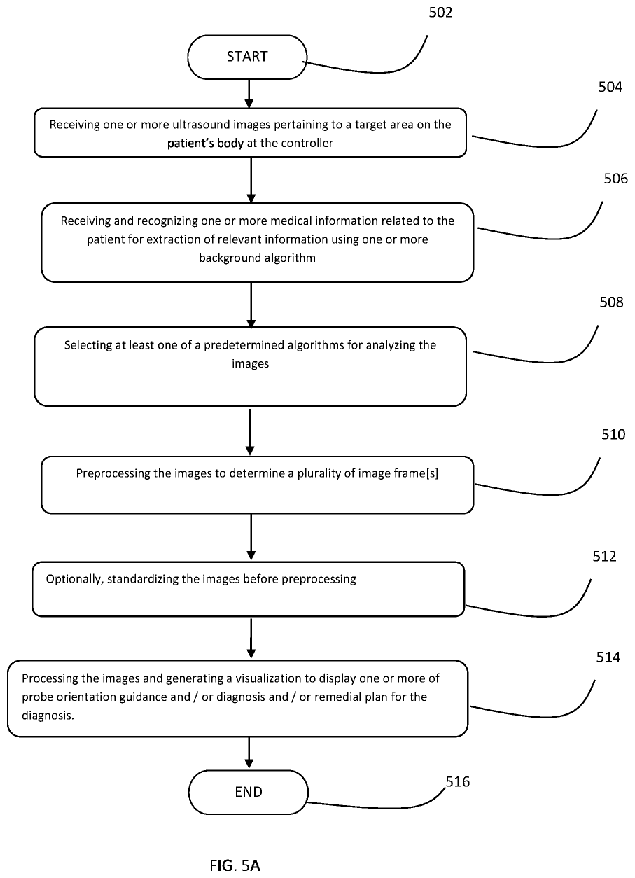

The system 100 further includes a visualization generation component 158 to generate a visualization of the probe orientation guidelines/diagnosis/remedial plans in accordance with the possibilities determined by one or more predetermined analysis algorithms 124. In a preferred embodiment, the visualization generation component 158 is configured within the controller 120 adapted to visualize the output onto the reporting unit 126 as illustrated in FIG. 4A.

FIG. 4A & FIG. 4B illustrate measurement marks indicating the scale dimensions of the image represented. A set of instructions uses character recognition and image recognition to allow method and system to automatically calculate the number of pixels between the measurement marks. This new calibration of information may determine the number of pixels per centimeter which may then be saved and used by the algorithms and reporting instructions on our method and system intention. FIG. 4B further illustrates a background application, a portable ultrasound device including but not limited to smart phone, tablet, which overlays an interactive button that when pressed in the foreground application opens a context menu for interacting with the background application. This allows for starting and stopping of recording and processing without leaving the foreground application. The same screen overlay process can be used to highlight key features or guide the ultrasound user to move the probe, depending on which algorithm and process is selected in the background application. The overlay button is not required, and the background application can be initiated prior to starting the ultrasound procedure with the patient. The background application screen, when brought to the foreground, in this embodiment is the same as the interactive screen in FIG. 4A.

In some embodiments, the visualization may include a time-based slider that may enable users to seamlessly switch between live and historical analysis/streams that can come from various sources (e.g., real-time store, temporary data cache, historical data store, etc.). Further, the real-time images may be compared with a historical baseline based on simultaneously streaming from a real-time ultrasound devices probe, a temporary data cache, or a historical data store. It is understood that various features (e.g., components, operations, or other features) described herein may be implemented separately or in combination with other features.

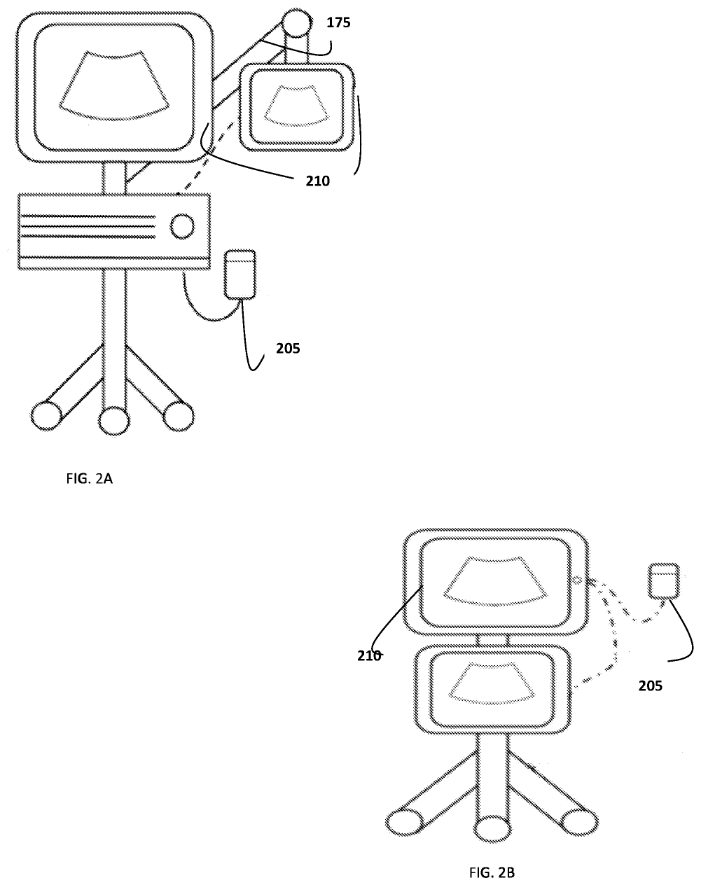

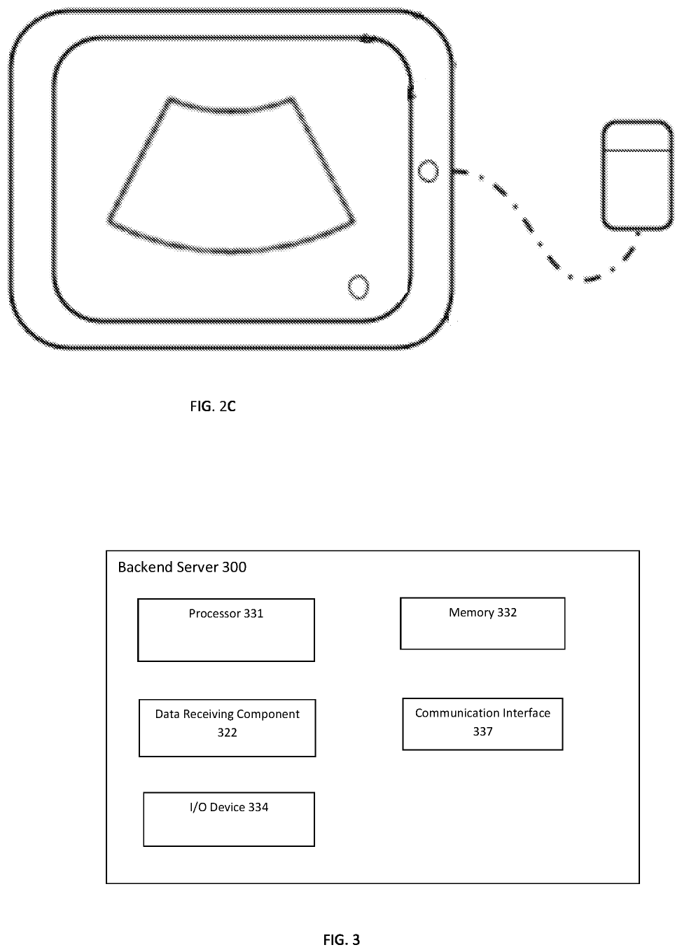

The ultrasound device 105 is intended to represent various forms of portable ultrasound scanning system, which includes multiple components that may be coupled to one another to form a single structure, may be separate but located within a common room, or may be remotely located with respect to one another. FIG. 2A through FIG. 2B illustrates various exemplary ultrasound devices 200. In one of the embodiments, as illustrated in FIG. 2A, the ultrasound device 200 includes an ultrasound transducer 205, and one or more diagnostic monitors 210 for displaying the images scanned by the ultrasound device 200. In addition, the ultrasound device 200 may include additional sensors for enabling a consistent scan of a target area, Additionally, the ultrasonic transducer 205 may include one or more orientation sensors for sensing, receiving, and sending orientation related information such as the direction, angle, and rotation of the ultrasonic transducer in real time towards the controller 120.

The ultrasound devices 105, and the controller 120 may be positioned together using one or more positioning means. Such as for example, in some instances, where the ultrasound devices 105 are connected to the controller 120 via a wired communication interface 112 as illustrated in FIG. 2A, the ultrasound devices 105 may be adapted to be installed/positioned onto a clamping arm 175, which may further be supported onto the controller 120. In some other embodiments, the ultrasound devices 105 and/or controller 120 are supported on a positioning cart. In yet other instances, any possible suitable means of positioning ultrasound devices 105 and controller 120 together may be utilized. In other instances, where the ultrasound devices 105 are connected to the controller 120 via a wireless communication interface 112, it is not required to position and/or install the ultrasound devices 105 and the controller 120 together.

The components shown here, their connections and relationships, and their functions, are meant to be exemplary only, and are not meant to limit implementations described and/or claimed in this document.

In a preferred embodiment, as illustrated in FIG. 3, the back-end server 120 is a computing device 300 having a processor 331, memory 332, a storage device 333, a high-speed interface connecting to memory and high-speed expansion ports, and a low-speed interface connecting to low speed bus, one or more input/output (I/O) devices 334. Each of the components 331, 332, 333, 334 is interconnected using various busses, and may be mounted on a common motherboard or in other manners as appropriate.

The processor 331 may communicate with a user through control interface [not shown] and display interface coupled to a display. The display may be, for example, a TFT LCD (Thin-Film-Transistor Liquid Crystal Display) or an OLED (Organic Light Emitting Diode) display, or other appropriate display technology. The display interface may comprise appropriate circuitry for driving the display to present graphical and other information to a user. The control interface may receive commands from a user and convert them for submission to the processor 331. In addition, an external interface in the form of data-receiving component 322 may be provided in communication with processor 331, so as to enable near area communication of the back-end server 300 with other controllers 120 within the facility 150. External interfaces may provide, for example, for wired communication in some implementations, or for wireless communication in other implementations, and multiple interfaces may also be used.

The backend server 300 is shown as including the memory 332. The memory 332 may store the executable programming instructions 160. The executable programming instructions 160 may be stored or organized in any manner and at any level of abstraction, such as in connection with one or more applications, processes, routines, procedures, methods, functions, etc.

In one implementation, the memory 332 is a volatile memory unit or units. In another implementation, the memory 332 is a non-volatile memory unit or units. The memory 332 may also be another form of computer-readable medium, such as a magnetic or optical disk. In one implementation, a computer program product is tangibly embodied in an information carrier. The computer program product contains instructions that, when executed, perform one or more methods, such as those described above. The information carrier is a computer- or machine-readable medium, such as the memory, expansion memory, or memory on processor.

The instructions stored in the memory 332 may be executed by one or more processors, such as a processor 331. The processor 331 may be coupled to one or more input/output (I/O) devices 335.

In some embodiments, the I/O device(s) 334 may include one or more of a keyboard or keypad, a touchscreen or touch panel, a display screen, a microphone, a speaker, a mouse, a button, a remote control, a joystick, a printer, a telephone, or mobile device (e.g., a smartphone), a sensor, etc.

The back-end server 300 may communicate wirelessly with the communication interfaces 114 of the controllers 120 through a back-end communication interface 337. The back-end communication interface 337 may provide for communications under various modes or protocols, such as HTTPS, MQTT, sMQTT over WIFI, LAN, or GSM voice calls, SMS, EMS, or MMS messaging, CDMA, TDMA, PDC, WCDMA, CDMA2000, or GPRS, among others. Such communication may occur, for example, through radio-frequency transceivers. In addition, short-range communication may occur, such as using a Bluetooth, Wi-Fi, or other such transceiver (not shown). However, in other embodiments, the data receiving component 322 may use one or more application programming interface (API) connected to the controller 120 so as to receive datasets 145 there from in a format acceptable by the source API and readable by the back-end server 140. An exemplary back-end server is depicted in FIG. 3.

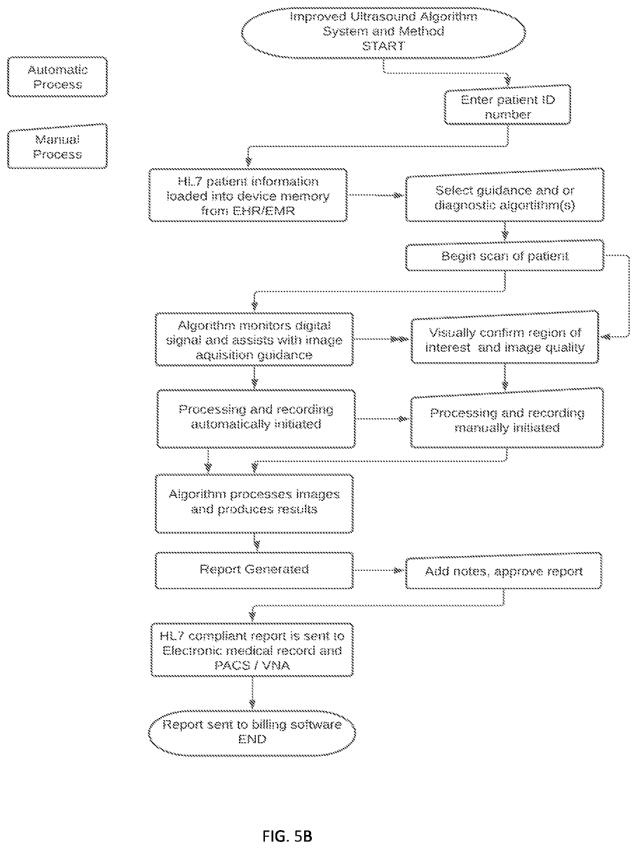

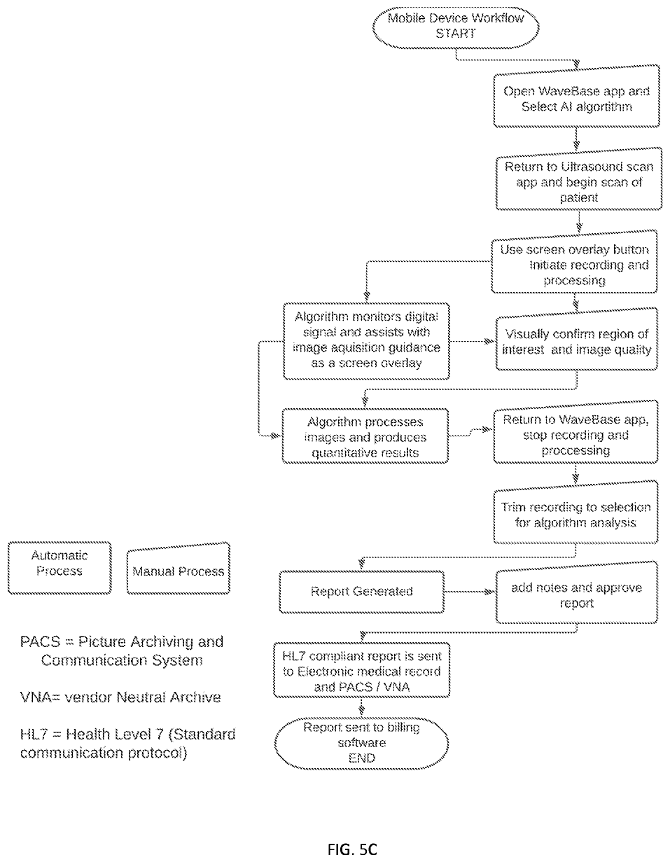

FIG. 5A illustrates a flow chart of a method of enhancing image acquisition and improving the diagnosis in real time, according to the present invention. The method starts at step 502 where processing is initiated either automatically or by a user input and method prior to step 504.

At step 504, one or more ultrasound images, pertaining to a target area on the patient's body, captured by one or more ultrasound devices 105, are received at the controller 120.

At step 506, the controller 120 further receives one or more medical information related to the patient where it is recognized for extraction of relevant information related to the patient using one or more background algorithms, such as an optical character recognizing component of the controller 120. In some embodiments, the medical information may be supplemented with additional information such as including but not limited to diagnostic probabilities, confidence percentage, highlighting, measuring, and annotating key features of the images.

At step 508, the controller 120 is configured to select at least one of a predetermined algorithm 124 out of the plurality of predetermined analysis algorithms 124 for analyzing the images and/or frames thereof. Such a selection may either be performed on the basis of the user's input in a manual mode or otherwise automatically when the system 100 is running in an automatic mode. In yet other embodiments, the controller 120 may suggest one of the algorithms 124, which still needs to be selected manually by the user. Further, in such embodiments, the controller 120 may select one or more additional algorithms for performing multiple tasks, either in combination or otherwise sequentially one after the other, or otherwise in any other possible order, as may be applicable. In a preferred embodiment, the step of analysis includes processing the images and/or frames thereof to perform a classification to define the target area, perform identification in comparison with the normal and/or historical data and other data sets 167 followed by diagnosing using CAD functions to prepare an output of the analysis.

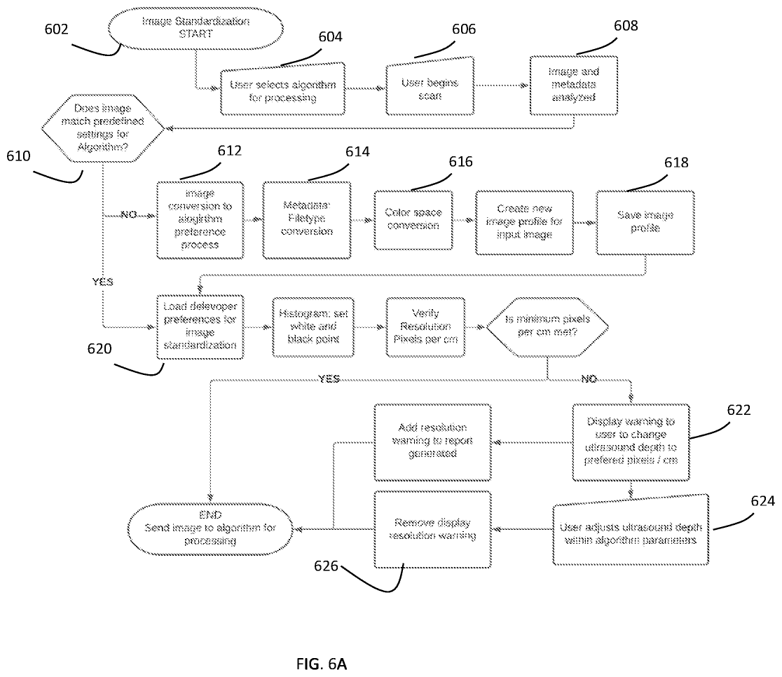

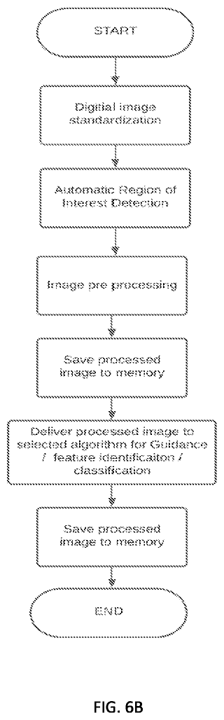

The method 500 further includes one or more optional steps before the step 508, for example, a step 510, where the images are preprocessed to determine a plurality of image frame[s] and or masking the images if required. Further, the pixels of the image frames are extracted and are possibly saved within one or more pixel-table adapted to store entries for a plurality of scanning depths, each of the entries in the table relates to a predetermined feature of an ultrasound image frame. In some embodiments, such extracted pixels may be stored in the internal storage 127 and may be used for future usage. In some embodiments, the step of image standardization may be performed by a method 600 as illustrated in FIG. 6A. The method 600 starts at step 602 and proceeds to step 604 where an algorithm is selected for starting an ultrasound scan at step 606. The method proceeds to step 608 where the scanned image is received at the controller 120 and is matched against a metadata stored within an internal storage of the controller to determine if the received image is in accordance with one or more predefined settings at step 610. If the received images are not in accordance with the predefined settings, the method proceeds to step 612 where the images are converted in accordance with an algorithm for converting the images for predefined settings which is then converted to metadata file type at 614 preceded by step 616 where color space is converted for the received image and is then saved at step 618. If, however, at step 610 it is determined that the image is in accordance with the predefined settings, the method proceeds to step 620 where the image is amended in accordance to preferred standardization and/or converted into a histogram and/or resolutions is verified in accordance with a predetermined pixel per centimeter. If the pixels per cm is in accordance with the preferred values, the image is considered as standardized. If not, the method proceeds to step 622 where a warning is generated for the user to change ultrasound depth in accordance with the pixel per cm followed by a rescan by the user at step 624 and the warning is removed at step 626 and the image is considered as standardized. If, however, the user does not rescan at step 624, the method proceeds directly from step 622 to step 626 where report is generated with the warning reported onto the image itself.

The method 500 furthermore includes an additional optional step 512, where images are standardized before preprocessing the images, for example, the images are processed in accordance with one of the algorithms so as to convert them into an industry standard data format (such as Health Level 7). Further, optionally the images in standard format may be stored in the Picture Archive and communication system [not shown] belonging to the controller 120 and/or otherwise remote to the controller in any third-party system.

Lastly, at step 514, the images are processed, and a visualization is generated to display one or more of probe orientation guidance and/or diagnosis and/or remedial plan for the diagnosis, in accordance to predetermined programming instructions 150 of the selected one or more algorithms 124. Accordingly, the visualization may be in the form of guidance feedback to the user about the direction and angle of the probe. Further, algorithms can indicate when an ultrasound image is properly acquired in the monitor which indicates to the user when to begin recording and/or save images. The images, processed output, and/or decisions are collated together in the form of a report, which may either be deleted, saved for later, modified and/or stored within the data repository 148 of the back-end server 140 or otherwise within the controller's inbuilt storage 127 and/or any Picture Archive and communication system. The process terminates at step 516.

FIG. 5B represents an exemplary flowchart according to an embodiment of the present invention. The process starts with patient's information such as ID number, wherein HL7 patient information is loaded into the device memory from HER/EMR. In the next step, the guidance and/or diagnostic algorithm is selected based on the patient's information and/or user input to the system. The patient scanning is started wherein the algorithm monitors digital signal thereby assisting with image acquisition guidance. The region of interest and image quality is visually confirmed. Further, the processing and recording of the image is automatically initiated. Further, optionally if required, manual processing or recording of the image may be performed by the user. The output generated based on the image processing is used for generating report, wherein the notes are added to the report and is then approved. The HL7 compliant report is sent to Electronic Medical record and PACS/VNA. The report is further sent to the billing software.

FIG. 5C further illustrates an exemplary flowchart for mobile device workflow, wherein an application is used/opened, and an AI algorithm is selected. The process is same as illustrated in FIG. 5B, however, once the image is processed and quantitative results are produced, the step is returned to the application.

FIG. 5B represents only six manual processes. Further, as illustrated, if the algorithm selected has the capabilities to guide the user and begin recording automatically when a clear image of the desired area is identified, only four manual processes will be retained thereby increasing the efficiency. FIG. 5C illustrates an ultrasound mobile device workflow to the standard use of algorithms, which represents seven manual processes only. The mobile application method and system illustrated herein includes five manual tasks when automatic guidance and recording are enabled. In addition to reducing the number of tasks for the user, the goal of these improved workflows is to improve reporting accuracy and consistency by removing manual steps which can be executed differently by different users.

In instances, where the system 100 is operated on the basis of a user's input, the system may utilize one or more input unit 117 such as a mouse, a keyboard and/or a microphone to interact with a user interface of the controller 120. In other embodiments, the user inputs may be input directly from the touchscreen interface on the reporting unit 126. Examples of user interaction include region of interest selection, measurement calibration, starting and stopping processing, starting, and stopping recording, annotating the images with drawings, text from the keyboard or touchscreen or speech to text. The user interface will also be used to add to and/or modify the reports generated & organized by the predetermined analysis algorithm. Further, in such embodiments, the additional information can be displayed in user editable fields such as for example Scan settings and display information such as the selected algorithm, pixel resolution and pixel to cm measurement information, and the like.

According to an embodiment, the controller 100 is exemplified with a client architecture system where the controller 120 may be in the form of a mobile application. The mobile application in such instances, includes a front-end user interface that can run off a standard web-browser on desktop environments, or a mobile based smartphone or tablet versions (for Android and iOS); and a backend server 140 which can be a lightweight workstation machine that will collect and process the datasets received from one or more controllers 120.

The mobile application displays different dashboards based on the type of the output to be displayed by the report generation component. The orientation feedback guidance alerts of any orientation error in accordance with the medical information.

Advantageously, such an accurate and timely assessment of ultrasound images is particularly beneficial in avoiding any faulty orientation/probe and/or diagnosis using the system 100 of the current disclosure. Further, the system 100 connects the physical and digital worlds by automating, collecting, and storing critical data, creating frictionless workflows to automate medical image processing.

Moreover, since the system 100 of the present subject matter is able to communicate via various possible communication interfaces known in the art, it provides flexibility to the organizations/facilities to choose the technology backhaul dependent on existing site infrastructure or requirements. Therefore, an infrastructure upgrade within the facility is not required.

FIG. 6B is a flowchart that outlines one embodiment of the system and method that allows images from different ultrasound manufacturers to be standardized and pre-processed to allow for algorithms that were originally designed and tested on one make and model of ultrasound imaging device to function as expected when using images taken from a different ultrasound device. Digital image standardization ensures that all digital image data including the frame rate, resolution, color format and image format are in the correct format for the algorithm selected. This set of instructions, when executed in a computer readable format enable the method and system outlined in this patent so that the algorithms, no matter who created them, can work with any input digital signal, no matter the manufacturer of the ultrasound device.

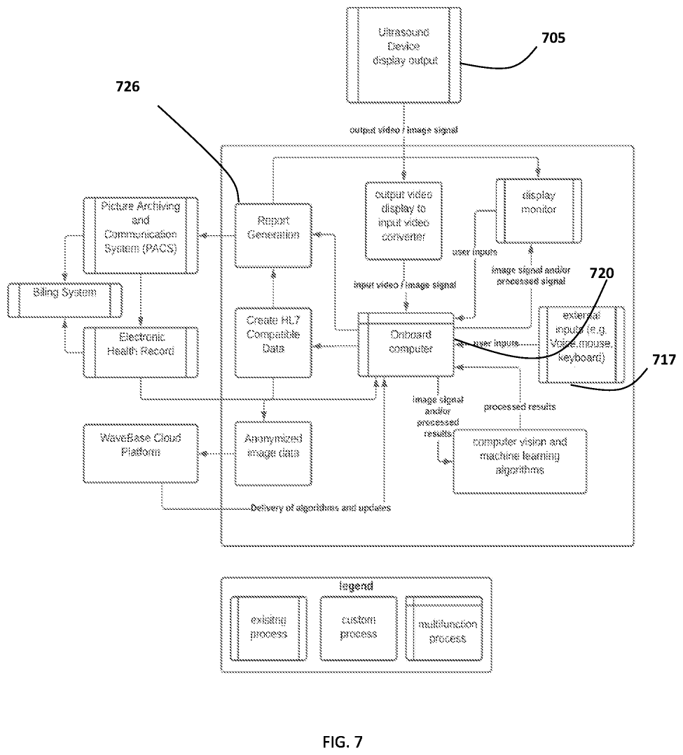

FIG. 7 Illustrates an exemplary working environment depicting the usage of system 700 in a practical situation. In such an exemplary embodiment, the system 700 includes an ultrasound device 705 for generating an image output which is displayed onto an ultrasound's output display and sent to the controller 720. The controller 720 further receives additional information such as medical information from the user input 717 in addition to processed datasets 145, 167 from the back-end server (not shown). The controller 720 processes the received images and generates a report which is displayed onto the reporting unit 726. Further, such reports are saved into external sources such as, for example, PACS (Picture Archive and Communication Systems, Vendor Neutral Archives), and, Electronic Health Records, which may be sent to the billing system as well.

The invention improves upon other approaches by providing a very simple level of integration that does not require a formal partnership with the manufacturer of the medical image device. Further, this allows the invention to work with any medical imaging device that permits the real time transmission of image data either wirelessly or via a wired connection. This user-installable, device diagnostic approach has benefits to users who are already familiar with a similar device and would like to add on computer vision and machine learning capabilities without the high costs of replacing the ultrasound imaging devices

The invention is a unique method and system that utilizes existing medical image data designed to be output for a secondary viewing monitor and instead delivers that data to a computer for real time computer vision analysis that adds features and outputs useful data to the user in real time. This goal can be achieved in several ways. Such as, but not limited to inputting the image data via a display output to universal serial bus (USB) converter, a direct integration of the graphics card to or a display output to camera serial interface or by wireless transmission. The receiving computer can be a desktop, laptop, mobile device, single board computer or system on a chip.

It is noted that various connections are set forth between elements in the description and in the drawings (the contents of which are included in this disclosure by way of reference). It is noted that these connections in general and, unless specified otherwise, may be direct or indirect and that this specification is not intended to be limiting in this respect. In this respect, a coupling between entities may refer to either a direct or an indirect connection.

Various embodiments of the invention have been disclosed. However, it should be apparent to those skilled in the art that modifications in addition to those described, are possible without departing from the inventive concepts herein. The embodiments, therefore, are not restrictive, except in the spirit of the disclosure. Moreover, in interpreting the disclosure, all terms should be understood in the broadest possible manner consistent with the context. In particular, the terms “comprise” and “comprising” should be interpreted as referring to elements, components, or steps, in a non-exclusive manner, indicating that the referenced elements, components, or steps may be present, or utilized, or combined with other elements, components, or steps that are not expressly referenced.

The disclosed methods and systems, as illustrated in the ongoing description or any of its components, may be embodied in the form of a computer system. Typical examples of a computer system include a general-purpose computer, a programmed microprocessor, a micro-controller, a peripheral integrated circuit element, and other devices, or arrangements of devices that are capable of implementing the steps that constitute the method of the disclosure.

The computer system comprises a computer, an input device, a display unit, and the Internet. The computer further comprises a microprocessor. The microprocessor is connected to a communication bus. The computer also includes memory. The memory may be Random Access Memory (RAM) or Read Only Memory (ROM). The computer system further comprises a storage device, which may be a hard-disk drive or a removable storage drive, such as, a floppy-disk drive, optical-disk drive, and the like. The storage device may also be a means for loading computer programs or other instructions into the computer system. The computer system also includes a communication unit. The communication unit allows the computer to connect to other databases and the Internet through an input/output (I/O) interface, allowing the transfer as well as the reception of data from other sources. The communication unit may include a modem, an Ethernet card, or other similar devices, which enable the computer system to connect to databases and networks, such as, LAN, MAN, WAN, and the Internet. The computer system facilitates input from a user through input devices accessible to the system through an I/O interface.

In order to process input data, the computer system executes a set of instructions that are stored in one or more storage elements. The storage elements may also hold data or other information, as desired. The storage element may be in the form of an information source, or a physical memory element present in the processing machine.

The programmable or computer-readable instructions may include various commands that instruct the processing machine to perform specific tasks, such as steps that constitute the method of the disclosure. The systems and methods described can also be implemented using only software programming or using only hardware or by a varying combination of the two techniques. The disclosure is independent of the programming language and the operating system used in the computers. The instructions for the disclosure can be written in all programming languages including, but not limited to, “C”, “C++”, “C #”, “C+”, “Embedded C”, “Visual C++,” Java”, “Python” and “Visual Basic”. Further, the software may be in the form of a collection of separate programs, a program module containing a larger program or a portion of a program module, as discussed in the ongoing description. The software may also include modular programming in the form of object-oriented programming. The processing of input data by the processing machine may be in response to user commands, the results of previous processing, or from a request made by another processing machine. The disclosure can also be implemented in various operating systems and platforms including, but not limited to, “i0S”, “Mac” “Unix,” “DOS,” “Android,” “Symbian,” and “Linux.”

The programmable instructions can be stored and transmitted on a computer-readable medium. The disclosure can also be embodied in a computer program product comprising a computer-readable medium, or with any product capable of implementing the above methods and systems, or the numerous possible variations thereof.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor and can be implemented in a high-level procedural and/or object-oriented programming language, and/or in assembly/machine language. As used herein, the terms “machine-readable medium” and “computer-readable medium” refer to any computer program product, apparatus and/or device (e.g., magnetic discs, optical disks, memory, Programmable Logic Devices (PLDs)) used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

A person having ordinary skills in the art will appreciate that the system, modules, and sub-modules have been illustrated and explained to serve as examples and should not be considered limiting in any manner. It will be further appreciated that the variants of the above disclosed system elements, or modules and other features and functions, or alternatives thereof, may be combined to create other different systems or applications.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), and the Internet.

The claims can encompass embodiments for hardware, software, or a combination thereof.

Although few implementations have been described in detail above, other modifications are possible. Moreover, other mechanisms for performing the systems and methods described in this document may be used. In addition, the logic flows depicted in the figures may not require the particular order shown, or sequential order, to achieve desirable results. Other steps may be provided, or steps may be eliminated, from the described flows, and other components may be added to, or removed from, the described systems. Accordingly, other implementations are within the scope of the following claims.

We claim:

1. A system for enhancing capture and reporting of an ultrasound device in real-time, the system comprising:
one or more ultrasound devices configured to generate an ultrasound image; and
a controller adapted to be connected to one or more ultrasound device through one or more connection medium, the controller comprising:
an input unit adapted to receive one or more medical information related to a patient,
a memory comprising one or more pixel-tables adapted to store entries for a plurality of scanning depths, each of the entries in the table relates to a predetermined feature of an ultrasound image frame,
a processing unit for processing one or more image frame(s) in accordance with one or more of a plurality of predetermined analysis models, each of the predetermined analysis models comprising one or more programming instructions embodied thereon the controller, and
a reporting unit adapted to display an output of the analysis, the output comprising one or more of device direction suggestions and/or a diagnosis report and/or a remedial plan;
the controller being configured to select one of a predetermined analysis model in accordance with the medical information of the patient to process the received image correctly and perform an analysis, the analysis comprising extraction of pixels from the images, calibration and processing thereof followed by storing into one or more pixel-tables.

2. The system of claim 1, wherein the connecting medium is a wired communication interface selected from one or more of but not limited to a USB, HDMI, and CSI.

3. The system of claim 1, wherein the connecting medium is a wireless communication interface selected from one or more of but not limited to picture archiving and communication systems, electronic health records, and vendor-neutral archives.

4. The system of claim 1, wherein the medical information comprising information related to the patient selected from one or more of but not limited to medical prescriptions, diagnosis guidelines and diagnosis history.

5. The system of claim 1, wherein the controller comprising an optical character recognizing component adapted to enable an extraction of information from the medical information.

6. The system of claim 1, wherein the reporting unit comprising one or more interactive touch screen monitors.

7. The system of claim 1 further comprising a clamping arm adapted to support one or more ultrasound devices on to the controller.

8. The system of claim 1, wherein the controller further comprising a network interface adapted to connect the controller with a back-end server

9. The system of claim 8, wherein the back-end server comprising a machine learning based image processing module adapted to receive images from a plurality of controllers and upgrade the predetermined analysis models thereof.

10. A method of enhancing capture and reporting of ultrasound devices in real time, the method comprising the steps of:
receiving at a controller, one or more ultrasound images captured by one or more ultrasound device, connected thereto;
receiving one or more medical information at the controller via an input unit; and
processing one or more image frame(s) of each of the received images, in accordance with one or more of a plurality of predetermined analysis models;
the controller being configured to select one or more predetermined analysis model in accordance with the medical information of the patient so as to process the received image correctly and perform an analysis, the analysis comprising extraction of pixels from the images, calibration and processing thereof followed by storing into one or more pixel-tables.

11. The method of claim 10, wherein the analysis comprising a step of preprocessing the images to determine a plurality image frames.

12. The method of claim 11, wherein the analysis optionally comprising a step of image standardization before the step of preprocessing.

13. The method of claim 11, wherein the analysis comprising processing the images by performing a classification and/or identification and/or computer aided diagnostic functions thereupon.

14. The method of claim 10, wherein the medical information comprising one or more of but not limited to medical prescription, diagnostic guidelines, and any other patient related information.

15. The method of claim 14, wherein the medical information is analyzed by a background application to extract relevant information.

16. The method of claim 10, wherein storing the plurality of extracted pixels corresponding to a scanning depth and a predetermined feature of the image frame within a memory of the controller;

17. The method of claim 10, further comprising upgrading each of the plurality of predetermined analysis models in accordance with a machine learning based image processing module at a back-end server, the back-end server communicatively connected to a plurality of controllers to receive a plurality of images therefrom.

18. The method of claims 10 and 17, wherein selecting one or more algorithms comprising selecting at least one initial predetermined analysis model from a plurality of models using the machine learning based image processing module based on the received images and the medical information of the patient.

19. The method of claim 18, further comprising selecting one or more additional predetermined analysis model from a plurality of models using the machine learning based image processing module based on the received images and the medical information of the patient.
